(19) United States
(12) Patent Application Publication
(10) Pub. No.: US 2010/0145350 A1
(43) Pub. Date: Jun. 10, 2010

(54) Syringe Plunger Seal

(75) Inventor: Roy B. Bogert, Lincoln Park, NJ (US)

Correspondence Address: FISH & RICHARDSON P.C., P.O. BOX 1022, MINNEAPOLIS, MN, 55440-1022, US

(73) Assignee: Advanced Biomaterial Systems, Inc., Chatham, NJ (US)

(21) Appl. No.: 12/705,313

(22) Filed: Feb. 12, 2010

Related U.S. Application Data
(63) Continuation of application No. 11/252,515, filed on Oct. 17, 2005.
(60) Provisional application No. 60/688,629, filed on Jun. 7, 2005.

(51) Int. Cl. A61B 17/58

(52) U.S. Cl. 606/93

(57) ABSTRACT

A bone filler delivery device includes a syringe assembly. The syringe assembly has a barrel with a first opening for dispensing bone filler and a second opening adapted to receive a plunger. A plunger is adapted to mate with the barrel to define a bone filler dispensing chamber therein. The plunger can move through the barrel in an axial direction. A seal is coupled to the plunger and is adapted to seal against the barrel. The seal includes an annular body and an annular first lip that extends from the body in a first direction and is adapted to flex toward the barrel when the plunger is moved through the barrel toward the first opening.

### CROSS-REFERENCE TO RELATED APPLICATION

This disclosure claims the benefit of priority of U.S. Provisional Patent Application No. 60/688,629, filed Jun. 7, 2005, which is incorporated by reference.

### TECHNICAL FIELD

The present application relates to a syringe plunger seal and, more particularly, to a syringe plunger seal that is useful for high pressure delivery of viscous material, such as bone filler.

### BACKGROUND

Syringe assemblies have been used to deliver bone filler to patients. A typical syringe assembly includes a syringe barrel with an opening for material delivery at one end. A plunger assembly is adapted to pass through the syringe barrel and push the bone filler through that opening. A plunger seal is coupled to the plunger and is adapted to contact an inner surface of the barrel as the plunger is moved through the barrel. Typically, O-rings are used as plunger seals.

The opening in the syringe barrel for delivering bone filler delivery is typically small. In some implementations, an elongated bone filler delivery tube is coupled to the opening. Bone filler travels through the small opening, through the elongated delivery tube and to a treatment area in the patient's body.

Typically, high pressure is used to deliver bone filler through the small opening, through the elongated delivery tube and into the treatment area.

### SUMMARY OF THE INVENTION

The present disclosure relates to a syringe plunger seal that is well-suited for applications involving high pressure delivery of viscous materials, such as bone fillers. Incorporating the seal into the syringe of a bone filler delivery device enhances a user's ability to deliver a precisely metered amount of bone filler to a treatment area inside a patient's body. The seal provides excellent sealing capabilities in a dispensing chamber of a syringe regardless of whether the syringe plunger is being advanced through the syringe barrel (e.g., to deliver material to a treatment area) or retracted through the syringe barrel (e.g., to draw excess material back from the treatment area).

In one aspect, a syringe assembly for delivering bone filler is disclosed. The syringe assembly includes a barrel with a first opening through which bone filler can be delivered and a second opening for receiving a plunger. A plunger is adapted to mate with the barrel to define a bone filler dispensing chamber therein. The plunger also is adapted to move through the barrel in an axial direction. A seal is coupled to the plunger for sealing against the barrel. The seal includes an annular body and an annular first lip that extends from the body in a first direction. The annular first lip is adapted to flex toward the barrel to enhance the sealing effect on the dispensing chamber when the plunger is moved toward the first opening. In some implementations, the flexing may be caused, at least in part, by an elevated relative pressure inside the dispensing chamber when the plunger is advanced through the barrel.

In certain implementations, the seal also includes an annular second lip that extends from the body in a second axial direction. In those implementations, the annular second lip is adapted to flex toward the barrel when the plunger is moved through the barrel in an opposite direction (i.e., toward the second opening). In some implementations, the flexing may be caused at least partially by the relatively low pressure that is created inside the dispensing chamber when the plunger is retracted through the barrel.

In another aspect, a bone filler mixing and delivery device includes a mixing section adapted to mix components to form bone filler, a dispensing section with a syringe assembly that is adapted to dispense the bone filler and a valve that can be manipulated to open a flow path between the mixing section and the dispensing section. Examples of such devices are disclosed in U.S. patent application Ser. Nos. 10/438,471 and 10/637,908, which are incorporated by reference in their entirety.

The syringe assembly includes a barrel, plunger and a seal coupled to the plunger. The barrel has a first opening for dispensing the bone filler and a second opening to receive a plunger. A plunger can be inserted into the barrel via the second opening to define a dispensing chamber therein.

The seal has an annular body. An annular first lip extends from the body in a first direction and is adapted to flex toward the barrel when the plunger is advanced toward the first opening. An annular second lip extends from the body in a second direction and is adapted to flex toward the barrel when the plunger is moved toward the second opening inside the barrel.

In some implementations, one or more of the following advantages may be present. The amount of control that a user can exercise over the flow of material from a syringe may be enhanced. Such enhanced control might be particularly important in applications relating, for example, to kyphoplasty, vertebroplasty or other similar procedures that involve the therapeutic delivery of bone filler or other restorative biomaterials to a damaged area. Such procedures (and others) often require the treating physician to exercise extreme care to deliver precise amounts of restorative material (i.e., bone filler) to precise locations inside the patient's body. Certain implementations of the plunger seal and the techniques disclosed herein can enhance the control that physician has over delivery of restorative material (e.g., bone filler).

Additionally, the amount of leakage past a seal of a plunger can be minimized. Minimized leakage may, in some instances, improve the performance of fluid delivery systems, such as, a syringe in a bone filler delivery device.

Moreover, if a plunger seal is intended to be reused, the life of the plunger seal may be prolonged by implementing the techniques disclosed herein. Indeed, a seal implementing the features disclosed herein might, in some instances, include more material than a conventional plunger seal. Accordingly, its wear time might be longer. The plunger seal's longer life may indeed extend the operating life of the assembly (e.g., a bone filler mixing device) that incorporates the plunger seal.

Other features and advantages will be apparent from the following description, drawings and claims.

### BRIEF DESCRIPTION OF THE DRAWINGS

FIG. 1 is a side view of a syringe assembly.

FIGS. 2A and 2B are cross-sectional, side views of a plunger seal arrangement.

FIG. 3 is a side view of a plunger seal.

FIG. 4 is a cross-sectional, side view of a plunger seal.

FIG. 5 is a detail view of a portion of a plunger seal.

FIG. 6 is an exploded view of a plunger assembly.

FIG. 7 is a side view of a plunger assembly.

FIG. 8 is a partial cross-sectional, side view of a plunger assembly.

FIG. 9 is a front view of a plunger seal.

FIG. 10 is a side view of a plunger seal.

FIG. 11 is a cross-sectional side view of a plunger seal.

FIGS. 12A and 12B is a cross-sectional, side view of a partial plunger seal assembly.

FIGS. 13A and 13B illustrate a cross-sectional, side view of a partial plunger seal assembly.

FIG. 14 is a perspective view of a bone cement mixing and delivery device.

Like reference symbols refer to similar elements.

### DETAILED DESCRIPTION

The present disclosure relates to a seal for a syringe plunger that is particularly well suited for high pressure delivery of viscous materials, such as bone fillers. Typical bone fillers include bone cement based materials (e.g., polymethyl methacrylate (PMMA)), coral based materials (e.g., Hydroxyapatite), calcium sulfates, beta tricalcium phosphates, silica based materials (e.g., ceramics), and human demineralized bone matrices and polymers, such as biocompatible polylatic-co-glycolic acid (i.e., “plaga”).

When a bone filler, such as bone cement, is being delivered into a patient, it is usually highly viscous. Indeed, typical viscosities range between 2,500 and 300,000 centipoises. Bone filler may be delivered to the patient with a syringe assembly that includes a fairly narrow dispensing hole connected to a narrow and elongated delivery tube. High pressures may be required to deliver the highly viscous material (i.e., bone filler) through the narrow opening and the narrow and elongated delivery tube. Indeed, typical pressures range between 20 and 1,600 pounds per square inch (psi) inside the dispensing chamber of the syringe barrel during bone filler delivery. Implementations of the seal and sealing techniques disclosed herein are particularly well suited for such applications. They also may be used in other applications.

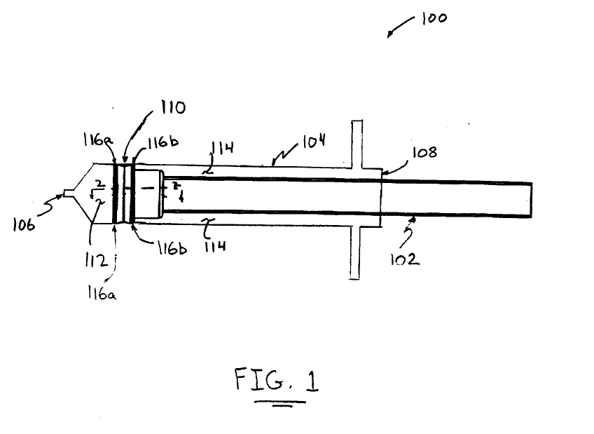

FIG. 1 illustrates a syringe assembly 100 that includes a plunger 102 partially positioned inside a syringe barrel 104. The syringe barrel 104 includes a first opening 106 for dispensing bone filler and a second opening 108 for receiving the plunger 102. The plunger 102 and the syringe barrel 104 define a dispensing chamber 112 that holds material (e.g., bone filler) that is available for dispensing. The inner diameter of the first opening is typically between about 2.5 and 4.5 millimeters. In one implementation, the inner diameter is about 3.2 millimeters.

An annular plunger seal 110 is coupled to the plunger 102 and is adapted to seal the dispensing chamber 112 from an area 114 behind the plunger seal 110. The plunger seal 110 includes flared annular lips 116a, 116b that extend from the seal 110 in approximately opposite axial directions. Each lip 116a, 116b is adapted to contact the inner surface of the barrel 104 and seal the dispensing chamber 112 from area 114. Each lip 116a, 116b also is adapted to flex in an approximately outward direction toward the barrel 104 when an appropriate force is applied. When such flexing occurs, the plunger seal's ability to prevent the passage of bone filler (and other material) between the dispensing chamber 112 and area 114 may be enhanced.

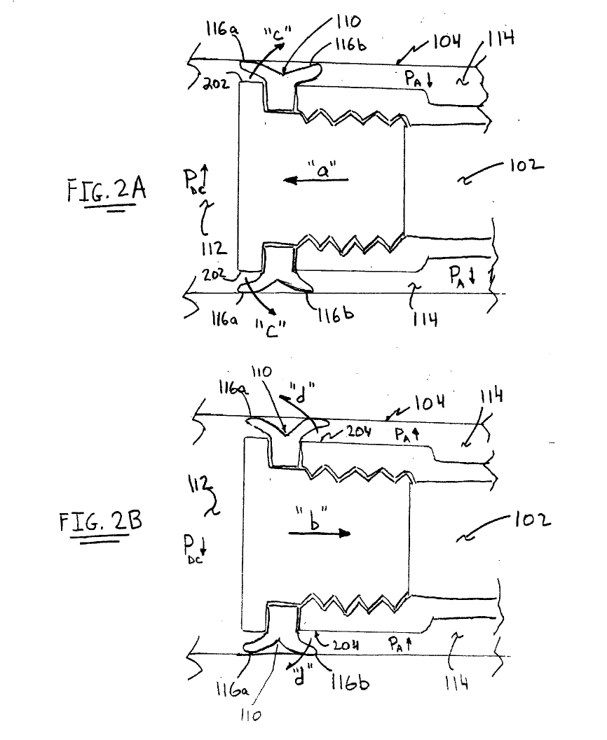

FIG. 2A is a cross-sectional, close up view of a plunger seal 110 area. According to the illustrated implementation, when the plunger 102 is advanced through the barrel 104 (i.e., moved in a direction indicated by the arrow marked “a”), the pressure (“P_{DC}”) inside the dispensing chamber 112 tends to increase and the pressure (“P_{A}”) in area 114 tends to decrease. The resulting pressure differential across the plunger seal 110 tends to cause the forward lip 116a to flex in an approximately outward direction against the barrel 104 (i.e., in approximately a direction indicated by the arrows marked “c”). Friction between the lip 116a and the barrel 104 as the plunger advances also may contribute to the lip's 116a flexing in the “c” direction. Also, bone filler from inside the dispensing chamber 112 may fill the space between the lip 116a and surface 202. That bone filler may contribute to flexing the lip 116a in the “c” direction. When the lip 116a flexes in the “c” direction, the seal's ability to prevent the passage of material from the dispensing chamber 112 to area 114 may be enhanced. That may be due, at least in part, to the contact area between the lip 116a and the barrel 104 being increased and the lip 116a being pressed firmly against the barrel 104 by a force resulting from the pressure differential across the lip 116a.

Additionally, as illustrated, the lip 116b on the trailing edge of seal 110 remains in contact with an inner surface of the barrel 104 as the plunger 102 is advanced through the barrel 104 in direction “a”. That trailing edge lip 116b provides a second sealing area (i.e., in addition to the sealing area created by the lip 116a contacting the barrel 104) between the dispensing chamber 112 and area 114, thereby further enhancing the sealing capability of the seal 110 as the plunger 102 is advanced.

FIG. 2B also is a cross-sectional, close up view of a plunger seal 110 area. According to the illustrated implementation, when the plunger 102 is retracted through the barrel 104 (i.e., moved in a direction indicated by the arrow marked “b”), the pressure (“P_{DC}”) inside the dispensing chamber 112 tends to decrease and the pressure (“P_{A}”) in area 114 tends to increase. The resulting pressure differential across the plunger seal 110 tends to cause the rear lip 116b to flex in an outward direction against the barrel 104 (i.e., in approximately the direction indicated by arrows “d”). Friction between the lip 116b and the barrel 104 as the plunger 102 is retracted also may contribute to the lip's 116b flexing in the “d” direction. Also, material from inside the area 114 may fill the space between lip 116b and surface 204 and push the lip 116b outward in the “d” direction. When the lip 116b flexes in the “d” direction, the seal's ability to prevent the passage of material (including air) from area 114 into the dispensing chamber 112 may be enhanced. That may be due, at least in part, to the contact area between the lip 116b and the barrel 104 being increased and by the lip 116b being pressed against the barrel 104 by a force resulting from the pressure differential across the lip 116b.

Additionally, as illustrated, the lip 116a on the trailing edge of the seal 110 tends to remain in contact with the barrel 104 as the plunger 102 is retracted. That trailing edge lip 116a provides a second sealing area (i.e., in addition to the sealing area created by the lip 116b being pressed against the barrel 104) between the dispensing chamber 112 and area 114, thereby further enhancing the sealing capability of the seal 110 as the plunger 102 is retracted.

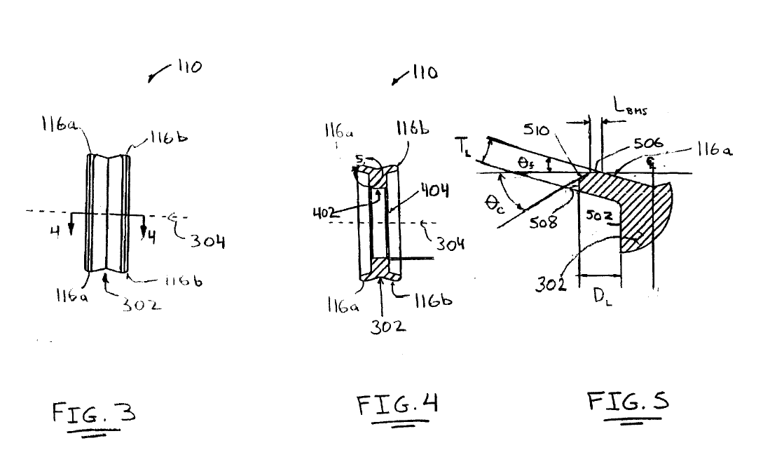

Referring now to FIGS. 3-5, the seal 110 has an annular body 302 that is formed about an axis 304. Annular first and second lips 116a, 116b extend from the body 302 in approximately opposite directions and are flared relative to the axis 304.

Typically, the seal 110 is made of a polymer material. According to one implementation, that polymer material is a polyvinyl difluoride. Other materials may be suitable for particular applications. For example, polyethylene, fluoropolymers, polypropylene and polyamides may be suitable in various implementations. The seal 110 has an internal surface 402 that defines an opening 404, through which a seal retaining screw or other mounting device can pass(see, e.g., FIGS. 2A and 2B). The opening 404 extends in an axial direction through the annular body 302.

According to the illustrated implementation, each lip 116a, 116b flares in an outward direction relative to the axis 304 of the seal 110 at an angle θ_{f}. Angle θ_{f} typically is provided to help ensure that the lips 116a, 116b will contact the barrel 104. In one implementation, the angle θₜ is between approximately 5° and 25°. More desirably, the angle θ_{f} is between approximately 10° and 20°. Still more desirably, the angle θ_{f} is approximately 15°.

Each lip 116a, 116b extends from the annular body 302 a distance D_{L}, Typically, that distance D_{L} is large enough to facilitate a desirable amount of lip deformation under expected operating pressures such that the sealing quality provided by the seal 110 can be improved by the lip being pressed against the barrel of the syringe. The appropriate distance D_{L} for a particular application may be influenced, for example, by the type of material used to manufacture the lips and/or the thickness of the lips. According to one implementation, the distance D_{L} (as measured from a vertically disposed and countersunk surface 502 of the seal to a tip 508 of lip 116a in an axial direction) is between approximately 0.05 and 0.07 inches. More desirably, that distance D_{L} is between approximately. 0.058 and 0.064 inches. Most desirably, that distance D_{L} is approximately 0.061 inches.

Generally, each lip 116a, 116b has a thickness T_{L} that allows it to deform in an outward direction under the influence of expected operating conditions. Typically, the inner diameter of a syringe barrel gradually decreases closer to its dispensing end (i.e., the end with the dispensing hole 106). Deciding how thick T_{L} each lip 116a, 116b should be may be influenced by the amount of deflection that the lips might experience as the plunger is advanced through the barrel's changing diameter. The appropriate thickness T_{L} for a particular application also may be influenced, for example, by the type of material used to manufacture the lips and/or the length of the lips. According to one implementation, the thickness T_{L} of each lip is between approximately 0.02 and 0.05 inches. More desirably, the thickness T_{L} is between approximately 0.032 and 0.038 inches.

As illustrated, each lip 116a, 116b includes a barrel mating surface 506 along an outer circumferential surface thereof. (See FIG. 5) As illustrated, the barrel mating surface 506 is largely cylindrical (i.e., not flared relative to the axis 304 of the seal 110) and is adapted to contact a corresponding approximately cylindrical inner surface of a syringe barrel during operation. In some implementations, the barrel mating surface 506 extends along the outer surface in an axial distance L_{BMS} for approximately 0.017 to 0.019 inches. More desirably, the barrel mating surface 506 extends approximately 0.018 inches. Instead of being cylindrical, in certain implementations, the barrel mating surface 506 may include flat portions, curved portions, sharp edges or other features.

Each lip 116a, 116b includes a chamfer 510 at a distal end of the barrel mating surface 506 that connects to an end 508 of the lip 116a, 116b. The chamfer 510 is provided to help prevent the seal 110 from catching on an edge of a barrel (e.g., barrel 104) when a plunger (e.g., plunger 102) is pushed into the barrel. As illustrated, the chamfer is disposed at an angle 6c relative to the barrel mating surface 506. According to one implementation, the angle θc is between approximately 15° and 45°. More desirably, the angle θc is approximately 30°.

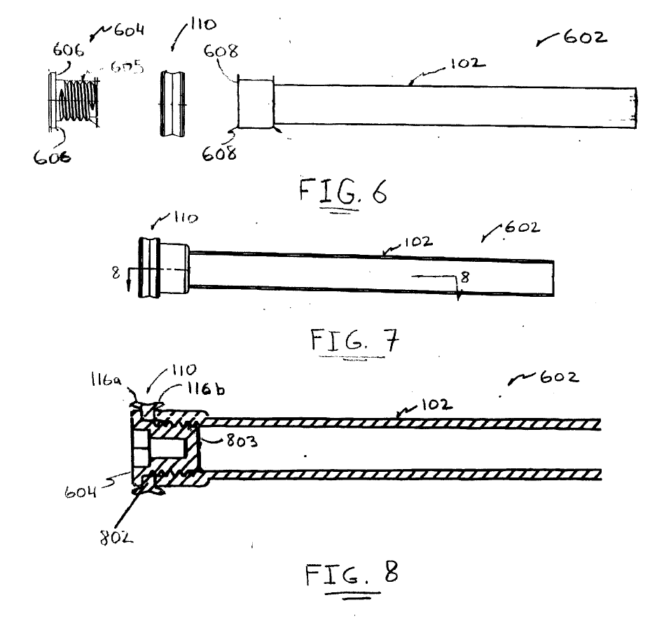

Referring now to FIGS. 6-8, a plunger assembly 602 includes a plunger seal 110 coupled to a plunger 102 and held in place by a seal retaining screw 604. The illustrated plunger 102 is a hollow tube that includes internal threads 803 adapted for mating with corresponding external threads 605 on the seal retaining screw 604. The seal retaining screw 604 and the plunger 102 include vertically disposed contact surfaces 606 and 608, respectively. The contact surfaces 606, 608 are adapted to contact opposite vertical surfaces of an inner portion 802 of seal 110. When assembled, the threaded section 605 of the seal retaining screw 604 passes through an annular opening in the seal 110 and engages the inner threads 803 of the plunger 102 and the contact surfaces 606, 608 contact opposing vertical surfaces on the inner portion 802 of the seal 110 to hold the seal 110 firmly in place. Lip 116a is adapted to extend from the annular body of the seal 110 over an end of the seal retaining screw 604. Lip 116b is adapted to extend from the annular body of the seal 110 over a portion of the plunger 102.

The illustrated plunger assembly 602 could be mated with a syringe barrel, such as syringe barrel 104. (See FIG. 1) Certain implementations of that arrangement might result in a syringe assembly adapted to minimize leakage past the plunger seal 110 during bone filler delivery and retraction. More specifically, if the plunger assembly 602 in such a device were advanced through the barrel 104 to deliver bone filler through an opening, the resulting pressure differential across the seal would tend to cause the first lip 116a on the plunger seal 110 to deflect toward the barrel 104, thereby enhancing the sealing strength of the seal 110. Similarly, if the plunger assembly is retracted from the barrel 104, an opposite pressure differential is created across the seal 110 that tends to cause the second lip 116b to deflect toward the barrel thereby enhancing the sealing strength of the seal 110.

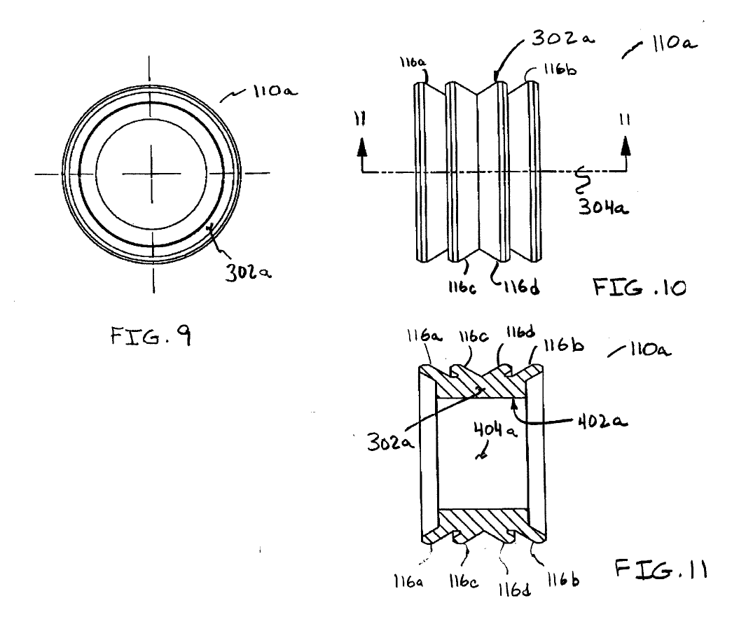

FIGS. 9-11 illustrate an implementation of a seal 110a that includes four lips 116a, 116b, 116c and 116d that extend from an annular body 302a formed about an axis 304a. Two of the lips (e.g., first lip 116a and third lip 116c) extend in approximately the same direction. The other two lips (e.g., second lip 116b and fourth lip 116d) extend in an approximately opposite direction. Each lip is flared relative to the axis 304a. The seal 110a has an internal surface 402a that defines an opening 404a, through which a seal retaining screw or other mounting device can pass. The opening 404a extends in an axial direction through the annular body 302a.

During operation, cooperating lips (e.g., 116a, 116c) may both deflect in a similar manner under the application of an appropriate force to provide a tight seal against the barrel of a syringe. Providing cooperating flexible lips (e.g., 116a, 116c) that extend and flex in substantially the same direction may provide enhanced sealing capabilities over a seal having only one flexible lip that extends in each particular direction.

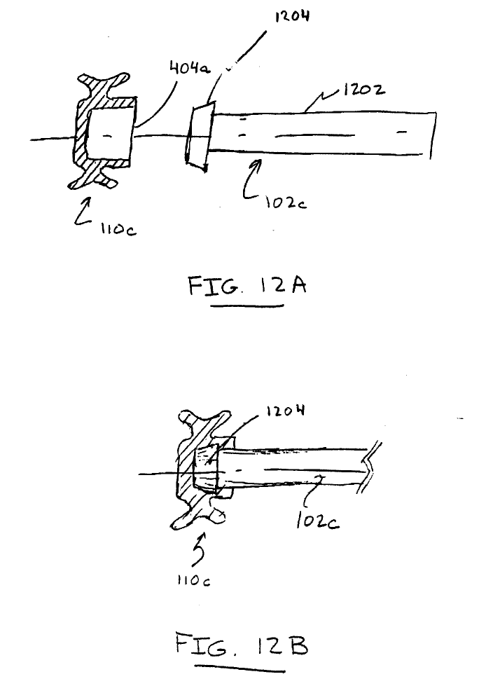

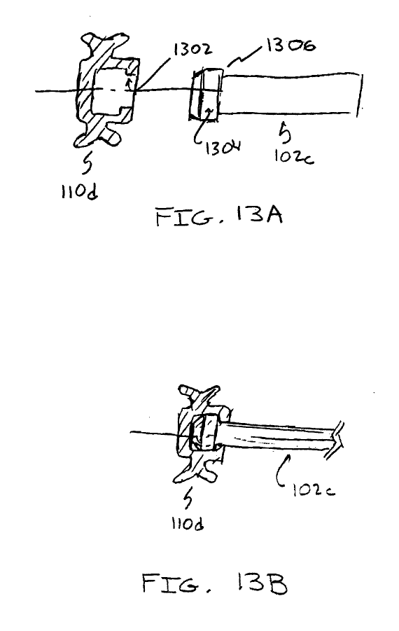

FIGS. 12A and 12B illustrate an alternative implementation a plunger seal 110c that can be mated to a plunger 102c using an interference fit. The illustrated plunger 102c includes a first portion 1202 and a second portion 1204. The first portion 1202 is a substantially cylindrical shaft. The second portion 1204 is at a forward end of the first portion 1202 and has an outer diameter that tapers from a larger diameter (where the second portion 1204 contacts the first portion 1202) to a smaller diameter (at a forward end of the first portion 1202).

The annular body of the plunger seal 110c defines an opening 404a that has a substantially cylindrical cross-section. The opening 404a extends partially through the body of the plunger seal 110c and bottoms at a substantially flat surface. The tapered outer diameter of the second portion 1204 facilitates fitting the second portion 1204 into the opening 404a in the plunger seal 110c.

As shown in FIG. 12B, the plunger seal 110c can be press fit over the second portion 1204 of the plunger 102c. When it is press fit in that manner, it can be held in place by friction.

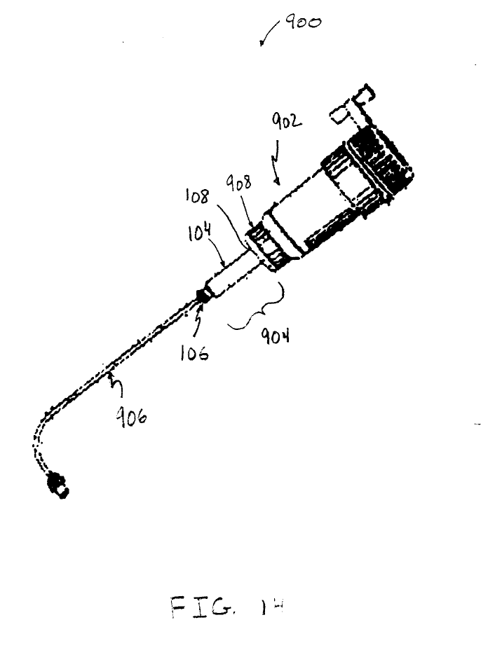

FIGS. 13A and 13B illustrate a plunger seal 110d that includes a gripping element 1302 adapted to engage a rear edge 1306 of the second portion 1304 of the plunger 102c. That engagement may, in some implementations, facilitate keeping the plunger seal 110d attached to the plunger 102c.

The apparatus and techniques disclosed herein may be incorporated into a syringe assembly of a bone cement mixing and delivery device, such as, the Plexis® bone cement mixing and delivery device, available from Advanced Biomaterial Systems, Inc. in Chatham, N.J. An example of a bone cement mixing and delivery device 900 incorporating the concepts and techniques disclosed herein is shown in FIG. 14. That device includes a mixing section 902 adapted to mix components to form bone cement and a dispensing section 904 with a syringe barrel 104 having an opening 106 at one end for dispensing mixed bone cement into a delivery tube 906. A valve 908 is positioned between the mixing section 902 and the dispensing section 904. The valve 908 can be manipulated to open a flow path between the mixing section 902 and the dispensing section 904.

The delivery tube 906 is a semi-flexible tube with an inner diameter that is typically between approximately 2.5 and 4.5 millimeters. In one implementation, the inner diameter is about 3.3 millimeters. The delivery tube 906 has a length between approximately ten and fifteen inches. In a particular implementation, the length is about ten inches long. The illustrated delivery tube 906 is bent.

The syringe barrel 104 has a second opening 108 that is mated to the valve 908 to receive a plunger (not shown, but positioned inside the device 900). The plunger is adapted to extend through the barrel 104 via the second opening 108 and toward the first opening 106 in the barrel 104. A seal incorporating the techniques disclosed herein is coupled to the plunger. The seal has an annular body with an axis and an annular first lip that extends from the body in a first direction that is flared relative to the axis. The seal also includes an annular second lip that extends from the body in a second direction and is flared relative to the axis.

According to one implementation, a user first mixes bone cement components in the mixing section 902 to form bone cement. After mixing, the user opens the valve 908 to allow the bone cement to flow into the dispensing section 904 (including the syringe barrel 104). Once the barrel 104 of the syringe contains an appropriate amount of bone cement, the user moves the plunger into engagement with the syringe barrel 104. When the plunger engages the syringe, a dispensing chamber is formed inside the syringe barrel 104. The user then advances the plunger through the barrel 104 toward the first opening 106. As the plunger advances, bone cement is pushed out of the first opening 106, through the delivery tube 906 and to a treatment area on a patient. The plunger seal helps to ensure that most of the bone cement in the plunger is delivered through the first opening 106 of the syringe barrel 104.

If the user determines that a sufficient amount (or even too much) of bone cement has been delivered to the treatment area, the user can begin to retract the plunger from the barrel 104. As the plunger is moved backwards through the barrel, the flow of bone cement at the outlet can be stopped and even reversed to some degree. The plunger seal assists in ensuring that the bone cement can be drawn back into the barrel 104, if needed.

The plunger seal helps provide flow control of bone cement through the syringe barrel 104.

A number of implementations have been described. Nevertheless, various modifications may be made without departing from the spirit and scope of the invention. For example, the design of each lip on a single seal need not be identical. One lip may be longer or thicker than the other. Additionally, various other materials may be suitable for different applications. The method of retaining the plunger seal in place may vary. One alternative arrangement may include a groove formed in the outer circumferential surface of the plunger that is adapted to receive the plunger seal and hold it in place. Additionally, the specific physical dimensions of the plunger seal may vary according to desired performance criteria. The plunger may include a counterbore on one, both or neither side. A counterbore may, for example, allow the seal retaining screw to be recessed into the plunger seal.

The plunger seal may be molded to an end of the plunger shaft.

The annular body of the plunger seal may have a non-cylindrical cross section. For example, the cross section may include one or more flat surfaces. Those flat surfaces may form a triangular, rectangular, pentagonal, hexagonal or other patterns.

Also, instead of including a chamfer at a distal end of the barrel mating surface, a radius could be provided. Such a radius could perform a similar function that the chamfer described above performs.

In certain implementations a seal may be provided that includes only one lip instead of two. Similarly, a seal may be provided having more than two lips that extend in a particular direction.

The plunger seal concepts disclosed herein could be adapted for use with any application in which a plunger passes through a barrel to move other highly viscous materials.

Accordingly, other implementations are within the scope of the following claims.

What is claimed is:

1. A syringe assembly for delivering bone filler, the syringe assembly comprising:
a barrel with a first opening through which bone filler can be dispensed and a second opening adapted to receive a plunger;
a plunger adapted to mate with the barrel to define a bone filler dispensing chamber in the barrel and adapted to move through the barrel in an axial direction; and
a seal coupled to the plunger for sealing against the barrel, wherein the seal comprises:
an annular body; and
an annular first lip that extends from the body in a first direction, wherein the annular first lip is adapted to flex toward the barrel and seal the dispensing chamber when the plunger is moved toward the first opening.

2. The bone filler delivery device of claim 1 wherein the annular first lip is adapted to flex toward the barrel when an elevated pressure inside the dispensing chamber exists.

3. The bone filler delivery device of claim 2 wherein movement of the plunger toward the first opening causes the elevated pressure inside the dispensing chamber.

4. The bone filler delivery device of claim 1 wherein the seal further comprises an annular second lip that extends from the body in a second direction, wherein the annular second lip is adapted to flex toward the barrel when the plunger is moved through the barrel toward the second opening.

5. The bone filler delivery device of claim 4 wherein the first direction is substantially opposite the second direction.

6. The bone filler delivery device of claim 5 wherein the first direction and the second direction are substantially axial directions.

7. The bone filler delivery device of claim 4 wherein the plunger is adapted to be retracted through the barrel away from the first opening, and wherein the plunger's retraction results in a pressure differential across the seal that tends to cause the second lip to flex toward the barrel.

8. The bone filler delivery device of claim 4 wherein the annular second lip is adapted to flex toward the barrel when a low pressure exists inside the dispensing chamber.

9. The bone filler delivery device of claim 4 wherein the annular first and second lips are flared relative to the axis.

10. The bone filler delivery device of claim 4 wherein, in a relaxed state, the annular first and second lips extend from the annular body in a direction that is displaced from the axis at an angle between approximately 5° and 25°.

11. The bone filler delivery device of claim 10 wherein, in a relaxed state, the first and second lips extend from the annular body in a direction that is displaced from the axis at an angle between approximately 10° and 20°.

12. The bone filler delivery device of claim 11 wherein, in a relaxed state, the first and second lips extend from the annular body in a direction that is displaced from the axis at an approximately 15° angle.

13. The bone filler delivery device of claim 4 wherein each of the first and second lips has a thickness that is between approximately 0.02 and 0.05 inches.

14. The bone filler delivery device of claim 4 wherein each of the first and second lips is approximately 0.032 to 0.38 inches thick.

15. The bone filler delivery device of claim 4 wherein each of the first and second lips includes a barrel mating portion that has a substantially cylindrical outer contour.

16. The bone filler delivery device of claim 15 wherein the barrel mating portion extends approximately 0.018 inches as measured in an axial direction.

17. The bone filler delivery device of claim 1 further comprising a bone filler delivery tube coupled to the first opening of the barrel and adapted to deliver bone filler to a patient.

18. The bone filler delivery device of claim 17 wherein the bone filler delivery tube has an inner diameter between approximately 2.5 and 4.5 millimeters and a length between approximately 10 and 15 inches.

19. The bone filler delivery device of claim 1 wherein the plunger is adapted to create a pressure inside the dispensing chamber up to approximately 1,400 to 1,800 pounds per square inch when it is moved toward the first opening inside the barrel.

20. The bone filler delivery device of claim 1 adapted for delivering bone filler having a viscosity between approximately 2,500 and 300,000 centipoises.

21. The bone filler delivery device of claim 1 wherein the plunger is adapted to be advanced through the barrel to dispense material through the first opening, and wherein the plunger's advancement results in a pressure differential across the seal that tends to cause the first lip to flex toward the barrel.

22. The bone filler delivery device of claim 1 wherein the seal comprises a polymer.

23. The bone filler delivery device of claim 1 wherein the seal comprises a polyvinyl difluoride.

24. The bone filler delivery device of claim 1 wherein the first lip includes a chamfer at a distal end thereof, wherein the chamfer is adapted to prevent the seal from catching on an edge of the second opening when the plunger is moved into engagement with the barrel.

25. The bone filler delivery device of claim 24 wherein the chamfer is disposed at an angle between approximately 15° and 45° relative to the axis.

26. The bone filler delivery device of claim 25 wherein the chamfer is disposed at an angle that is approximately 30° relative to the axis.

27. The bone filler delivery device of claim 1 wherein the bone filler is bone cement.

28. A bone cement mixing and delivery device, comprising:
a mixing section adapted to mix components to form bone cement;
a dispensing section with a syringe assembly that is adapted to dispense the bone cement; and
a valve that can be manipulated to open a flow path between the mixing section and the dispensing section;
wherein the syringe assembly comprises:
a barrel with a first opening for dispensing the bone cement and a second opening to receive a plunger;
a plunger that can be inserted into the barrel via the second opening to define a dispensing chamber therein; and
a seal coupled to the plunger, wherein the seal comprises an annular body, an annular first lip that extends from the body in a first direction and is adapted to flex toward the barrel when the plunger is advanced toward the first opening and an annular second lip that extends from the body in a second direction and is adapted to flex toward the barrel when the plunger is moved toward the second opening inside the barrel.

29. The bone cement mixing and delivery device of claim 28
wherein the annular first lip is adapted to flex toward the barrel when an elevated pressure inside the dispensing chamber exists; and
wherein the annular second lip is adapted to flex toward the barrel when a low pressure exists inside the dispensing chamber.

30. The bone cement mixing and delivery device of claim 28 wherein the annular first and second lips are flared relative to an axis of the plunger.

31. The bone cement mixing and delivery device of claim 28 further comprising a bone cement delivery tube coupled to the first opening of the barrel and adapted to deliver bone cement to a patient.

32. The bone cement mixing and delivery device of claim 28 wherein the bone cement delivery tube has an inner diameter between approximately 2.5 and 4.5 millimeters and a length between approximately 10 and 15 inches.

33. The bone cement mixing and delivery device of claim 28 adapted for delivering bone cement having a viscosity between approximately 2,500 and 300,000 centipoises.

34. The bone cement mixing and delivery device of claim 28 wherein the seal comprises a polymer.

35. The bone cement mixing and delivery device of claim 28 wherein the seal comprises a polyvinyl difluoride.

36. The bone cement mixing and delivery device of claim 28 wherein the seal further comprises:
a third annular lip that extends from the body in the first direction and is adapted to flex toward the barrel when the plunger is advanced toward the first opening; and
a fourth annular lip that extends from the body in the second direction and is adapted to flex toward the barrel when the plunger is moved toward the second opening inside the barrel.
